(19) United States
(12) Patent Application Publication
(10) Pub. No.: US 2021/0316129 A1
(43) Pub. Date: Oct. 14, 2021

(54) CATHETER ADAPTER PUSH TAB AND RELATED DEVICES, SYSTEMS, AND METHODS

(71) Applicant: Becton, Dickinson and Company, Franklin Lakes, NJ (US)

(72) Inventors: Yun Hui Wong, Singapore (SG); Kiat Jin Cheng, Singapore (SG)

(21) Appl. No.: 17/208,994

(22) Filed: Mar. 22, 2021

Related U.S. Application Data
(60) Provisional application No. 63/009,350, filed on Apr. 13, 2020.

(51) Int. Cl. A61M 39/10

(52) U.S. Cl. CPC A61M 39/10; A61M 2039/1077

(57) ABSTRACT

A catheter assembly may include a catheter adapter, which may include a distal end, a proximal end, and a lumen extending between the distal end and the proximal end. The catheter assembly may include a push tab. The push tab may be pivotally coupled to the catheter adapter. The push tab may be pivotally moveable between a raised position and a depressed position. The catheter assembly may include another push tab, which may be resilient and may include a first end, a second end, and an arced portion between the first end and the second end. The other tab push tab may be configured to move from a raised position to a depressed position.

### RELATED APPLICATIONS

This application claims the benefit of U.S. Provisional Patent Application No. 63/009,350 filed on Apr. 13, 2020, entitled CATHETER ADAPTER PUSH TAB AND RELATED DEVICES, SYSTEMS, AND METHODS, which is incorporated herein in its entirety.

### BACKGROUND

Infusion therapy, a common healthcare procedure, may be facilitated by a vascular access device. Hospitalized, home care, and other patients receive fluids, pharmaceuticals, and blood products via a vascular access device inserted into the vascular system. Blood withdrawal is another common healthcare procedure that may be facilitated by the vascular access device.

The vascular access device may access a peripheral or central vasculature of a patient. The vascular access device may be indwelling for short term (days), moderate term (weeks), or long term (months to years). The vascular access device may be used for continuous infusion therapy or for intermittent therapy.

A common type vascular access device is an over-the-needle peripheral intravenous catheter (PIVC). As its name implies, the “over-the-needle” PIVC may be mounted over an introducer needle having a sharp distal tip. The sharp distal tip may be used to pierce skin and the vasculature of the patient. Insertion of the PIVC into the vasculature may follow the piercing of the vasculature by the introducer needle. The introducer needle and the PIVC are generally inserted at a shallow angle through the skin into the vasculature of the patient with a bevel of the introducer needle facing away from the skin of the patient. Once placement of the introducer needle within the vasculature has been confirmed, the clinician may withdraw the introducer needle from the PIVC, leaving the PIVC in place for future fluid infusion and/or blood withdrawal.

The PIVC may extend from a catheter adapter, which may include a push tab to aid the clinician in inserting the PIVC into the vasculature. In some instances, securement tape may be used to secure the catheter adapter to skin of the patient. However, the push tab may result in tenting of the securement tape, which may result in improper securement of the catheter adapter to the patient and possible dislodgement.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a catheter adapter push tab, as well as related devices, systems, and methods. In some embodiments, a catheter assembly may include a catheter adapter, which may include a distal end, a proximal end, and a lumen extending between the distal end and the proximal end. In some embodiments, the catheter assembly may include a push tab, which may be pivotally coupled to the catheter adapter. In some embodiments, the push tab may be pivotally moveable between a raised position and a depressed position.

In some embodiments, the push tab may include a first end and a second end opposite the first end. In some embodiments, the first end may be pivotally coupled to the catheter adapter. In some embodiments, the second end may be further from the catheter adapter when the push tab is in the raised position than when the push tab is in the depressed position.

In some embodiments, the second end may be disposed in a first position in response to the push tab being in the raised position. In some embodiments, the second end may be disposed in a second position in response to the push tab being in the depressed position. In some embodiments, the second position may be proximal to the first position.

In some embodiments, the push tab may include two opposing protrusions. In some embodiments, the catheter adapter may include two opposing indents. In some embodiments, the two opposing protrusions may be disposed within the two opposing indents to pivotally couple the push tab to the catheter adapter.

In some embodiments, in response to the push tab being in the raised position, the push tab may be perpendicular or generally perpendicular to a longitudinal axis of the catheter adapter. In some embodiments, in response to the push tab being in the depressed position, the push tab may be generally aligned with the longitudinal axis of the catheter adapter. In some embodiments, the push tab may include an edge configured to contact the catheter adapter in response to the push tab pivotally moving from the depressed position to the raised position.

In some embodiments, a proximal surface of the push tab may be concave, which may facilitate securement of a digit of the clinician against the proximal surface. In some embodiments, the catheter adapter may include a side port disposed between the distal end and the proximal end of the catheter adapter. In some embodiments, the push tab may be pivotally coupled to the catheter adapter proximal to the side port.

In some embodiments, the catheter adapter may include a resilient push tab, which may be coupled to the catheter adapter. In some embodiments, the resilient push tab may include a first end, a second end, and an arced portion between the first end and the second end. In some embodiments, the resilient push tab may be configured to move from a raised position to a depressed position. In some embodiments, the arced portion may be further from the catheter adapter when the resilient push tab is in the raised position than when the resilient push tab is in the depressed position. In some embodiments, in response to the resilient push tab being in the raised position, the first end and the second end may be biased outwardly by the catheter adapter. In some embodiments, in response to the resilient push tab moving from the raised position to the depressed position, the first end and the second end may be brought closer together.

In some embodiments, the arced portion may contact the catheter adapter in response to the resilient push tab being moved from the raised position to the depressed position. In some embodiments, the resilient push tab may be coupled to the catheter adapter proximal to the side port.

In some embodiments, a method may include inserting a catheter assembly into vasculature of the patient. In some embodiments, the catheter assembly may include the catheter adapter, which may include the distal end, the proximal end, and the lumen. In some embodiments, the catheter assembly may include a push tab coupled to the catheter adapter. In some embodiments, the push tab may be moveable between a first position and a second position. In some embodiments, the push tab may be disposed in the first position.

In some embodiments, when the catheter assembly is inserted into the vasculature of the patient, the method may include pushing down on the push tab to move the push tab from the first position to the second position. In some embodiments, after pushing down on the push tab to move the push tab from the first position to the second position, the method may include applying securement tape over the push tab with the push tab in the second position.

In some embodiments, the push tab may be pivotally coupled to the catheter adapter. In some embodiments, the push tab may be pivotally moveable between the first position and the second position. In some embodiments, the first position may include the raised position. In some embodiments, the second position may include the depressed position. In some embodiments, the push tab may include the resilient push tab. In some embodiments, the arced portion may be further from the catheter adapter when the resilient push tab is in the first position than when the resilient push tab is in the second position.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, illustrating an example push tab in a raised position, according to some embodiments;

FIG. 1B is another upper perspective view of the catheter system, illustrating a digit of a clinician pushing on the push tab of FIG. 1A in the raised position during insertion of the catheter system into vasculature of the patient, according to some embodiments;

FIG. 1C is another upper perspective view of the catheter system, illustrating the push tab of FIG. 1A in the raised position, according to some embodiments;

FIG. 1D is an upper perspective view of the catheter system, illustrating an example needle assembly partially removed from an example catheter assembly, and the push tab of FIG. 1A in a depressed position, according to some embodiments;

FIG. 2A is a perspective view of the push tab of FIG. 1A, according to some embodiments;

FIG. 2B is another perspective view of the push tab of FIG. 1A, according to some embodiments;

FIG. 3A is an upper perspective view of the catheter assembly of the catheter system, illustrating the push tab of FIG. 1A removed for illustrative purposes, according to some embodiments;

FIG. 3B is an upper perspective view of the catheter assembly of the catheter system, illustrating the push tab of FIG. 1A removed for illustrative purposes, according to some embodiments;

FIG. 4A is an upper perspective view of the catheter system, illustrating another push tab disposed in a raised position, according to some embodiments;

FIG. 4B is an upper perspective view of the catheter assembly, illustrating the other push tab disposed in a depressed position, according to some embodiments;

FIG. 4C is an end view of the other push tab, according to some embodiments;

FIG. 4D is a proximal end view of the catheter assembly, illustrating the other push tab in the raised position; and

FIG. 4E is a proximal end view of the catheter assembly, illustrating the other push tab in the depressed position, according to some embodiments.

### DESCRIPTION OF EMBODIMENTS

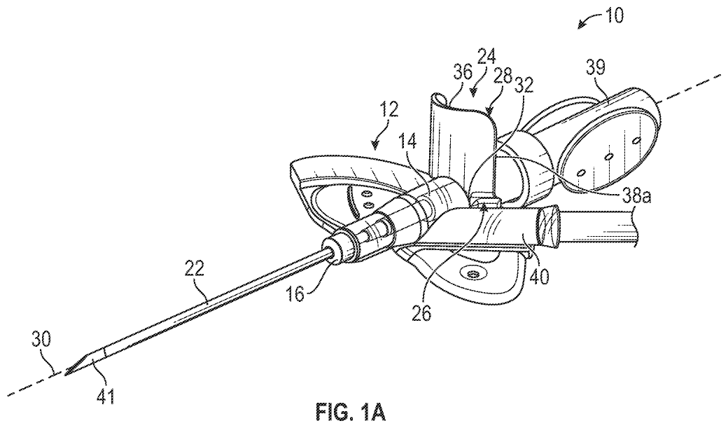

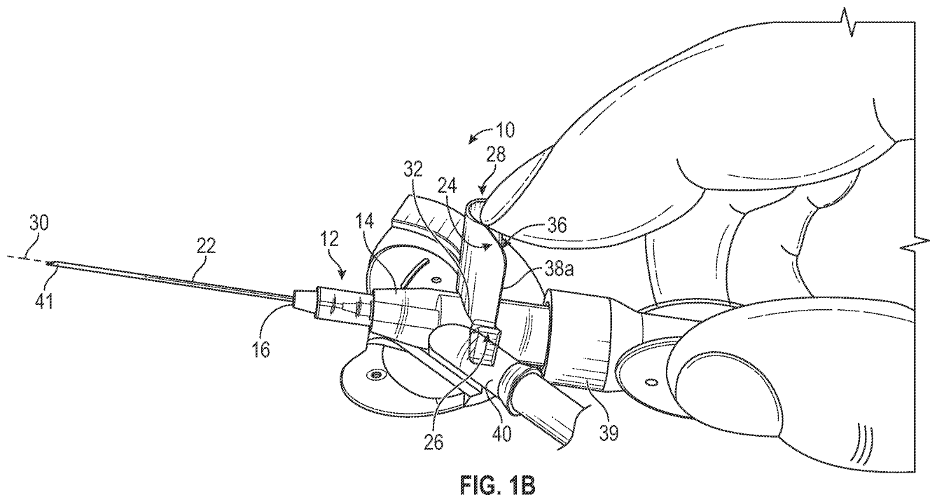

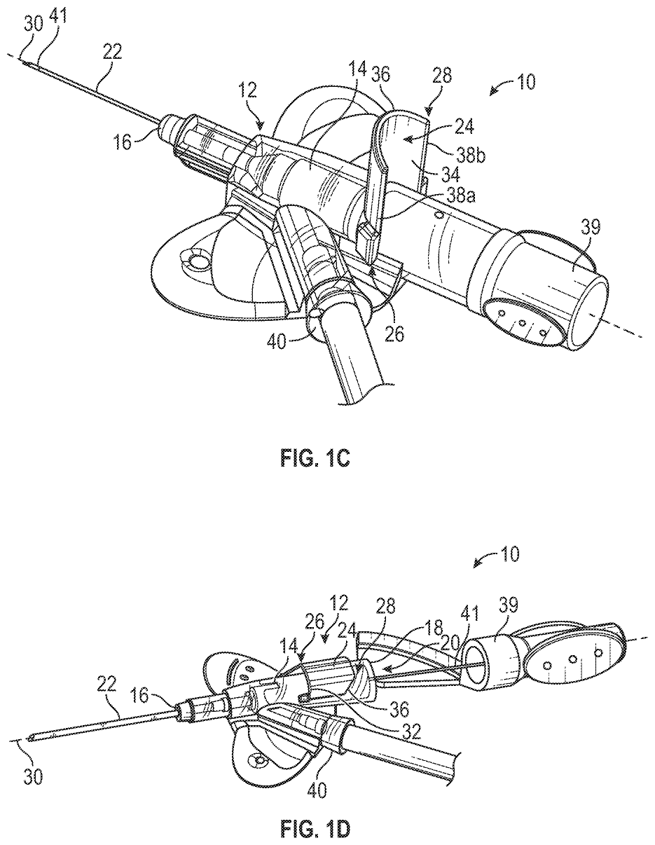

Referring now to FIGS. 1A-1D, an example catheter system 10 is illustrated, according to some embodiments. In some embodiments, the catheter system 10 may include a catheter assembly 12, which may include a catheter adapter 14. In some embodiments, the catheter adapter 14 may include a distal end 16, a proximal end 18, and a lumen 20 extending between the distal end 16 and the proximal end 18. In some embodiments, the catheter assembly 12 may include a catheter 22, which may be secured within the catheter adapter 14 and extend distally from the distal end 16 of the catheter adapter 14. In some embodiments, the catheter 22 may include a peripheral intravenous catheter (PIVC), a peripherally-inserted central catheter, or a midline catheter.

In some embodiments, the catheter assembly 12 may include a push tab 24, which may be pivotally coupled to the catheter adapter 14. In some embodiments, the push tab 24 may be pivotally moveable between a raised position, illustrated, for example, in FIGS. 1A-1C, and a collapsed or depressed position, illustrated, for example, in FIG. 1D.

In some embodiments, the push tab 24 may include a first end 26 and a second end 28 opposite the first end 26. In some embodiments, the first end 26 may be pivotally coupled to the catheter adapter 14. In some embodiments, the second end 28 may be further from the catheter adapter 14 when the push tab 24 is in the raised position than when the push tab 24 is in the depressed position.

In some embodiments, the second end 28 may be disposed in a first position in response to the push tab 24 being in the raised position, as illustrated, for example, in FIGS. 1A-1C. In some embodiments, the second end 28 may be disposed in a second position in response to the push tab 24 being in the depressed position, as illustrated, for example, in FIG. 1D. In some embodiments, the second position of the second end 28 may be proximal to the first position of the second end 28.

In some embodiments, in response to the push tab 24 being in the raised position, the push tab 24 may be perpendicular or generally perpendicular to a longitudinal axis 30 of the catheter adapter 14. For example, the push tab 24 may be angled between 45 degrees and 135 degrees or between 60 degrees and 120 degrees with respect to the longitudinal axis 30. In some embodiments, the push tab 24 be angled at 90 degrees with respect to the longitudinal axis 30, which may facilitate pushing by a digit of the clinician. In some embodiments, in response to the push tab 24 being in the depressed position, the push tab 24 may be aligned or generally aligned with the longitudinal axis 30 of the catheter adapter 14. In some embodiments, the push tab 24 may include an edge 32 configured to contact the catheter adapter 14 in response to the push tab 24 pivotally moving from the depressed position to the raised position. In some embodiments, the edge 32 may contact the catheter adapter 14 to prevent the push tab 24 from moving distal to the raised position. In some embodiments, the edge 32 may be arc-shaped.

In some embodiments, a proximal surface 34 of the push tab 24 may be concave, which may facilitate securement of a digit of the clinician against the proximal surface 34. In some embodiments, the proximal surface 34 may be bordered by the edge 32, another edge 36 opposite the edge 32, and two generally parallel edges 38a,b extending between the edge 32 and the other edge 36,

In some embodiments, the catheter adapter 14 may include a side port 40 disposed between the distal end 16 and the proximal end 18 of the catheter adapter 14. In some embodiments, the push tab 24 may be pivotally coupled to the catheter adapter 14 proximal to the side port 40. In some embodiments, an extension tube may be integrated with the side port 40. In some embodiments, the side port ′40 may be used for blood collection and/or fluid infusion. In some embodiments, the catheter adapter 14 may not include the side port 40.

In some embodiments, the push tab 24 may be positioned to facilitate one-handed insertion by the clinician into the vasculature of the patient. In some embodiments, a length of the push tab 24 between the first end 26 and the second end 28 may also be great enough to facilitate one-handed insertion by the clinician into the vasculature of the patient.

In some embodiments, the catheter system 10 may include a needle assembly, which may include a needle hub 39 and an introducer needle 41 extending through the catheter 22. In some embodiments, a proximal end of the introducer needle 41 may be secured within the needle hub 39. In some embodiments, the introducer needle 41 may include a sharp distal tip. In some embodiments, the needle hub 39 may include opposite side grips, which may facilitate the one-handed grip. In some embodiments, the needle assembly may be removed from the catheter system 10 in response to the catheter 22 being placed within the vasculature.

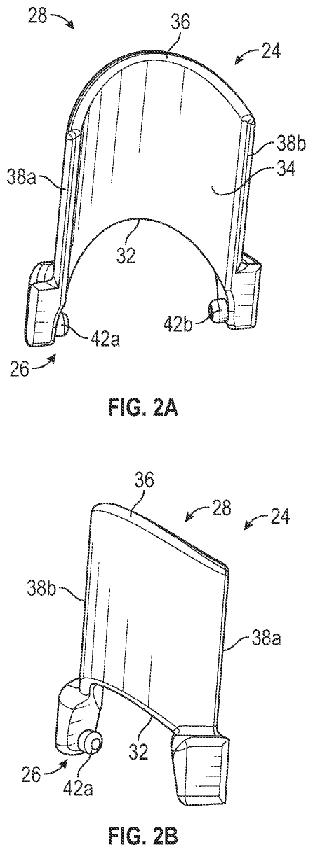

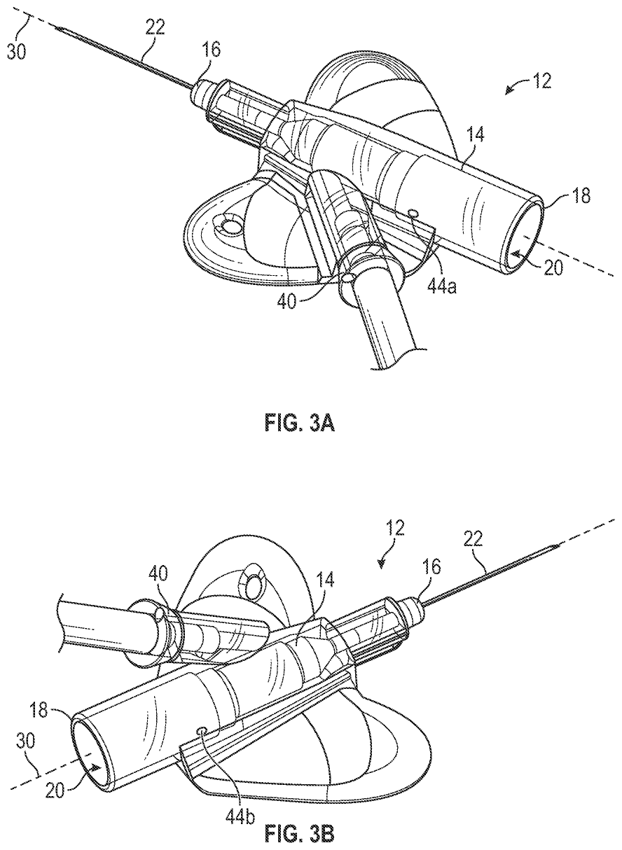

Referring now to FIGS. 2A-2B, in some embodiments, the push tab 24 may include two opposing protrusions 42a,b. Referring now to FIGS. 3A-2B, in some embodiments, the catheter adapter may include two opposing indents 44a,b. In some embodiments, the two opposing protrusions 42a,b may be disposed within the two opposing indents 44a,b to pivotally couple the push tab 24 to the catheter adapter 14.

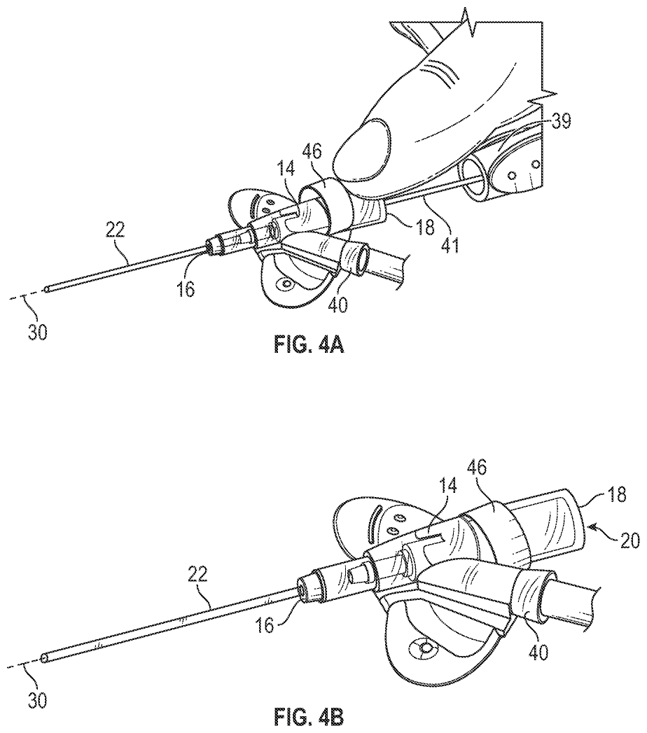

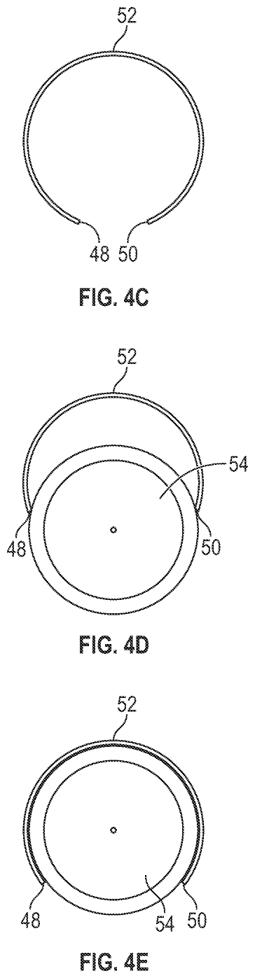

Referring now to FIGS. 4A-4E, in some embodiments, the catheter adapter 14 may include a resilient push tab 46, which may be coupled to the catheter adapter 14. In some embodiments, the resilient push tab 46 may include a first end 48, a second end 50, and an arced portion 52 between the first end 48 and the second end 50. In some embodiments, the resilient push tab 46 may be constructed of metal, an elastomer, or another suitable material.

In some embodiments, the resilient push tab 46 may be configured to move from a raised position, illustrated for example, in FIGS. 4A and 4D, to a depressed position, illustrated, for example in FIGS. 4B and 4E. In some embodiments, the arced portion 52 may be further from the catheter adapter 14 when the resilient push tab 46 is in the raised position than when the resilient push tab 46 is in the depressed position. In some embodiments, in response to the resilient push tab 46 being in the raised position, the first end 48 and the second end 50 may be biased outwardly from a resting position, illustrated, for example, in FIG. 4C, by the catheter adapter 14. In some embodiments, in response to the resilient push tab 46 moving from the raised position to the depressed position, the first end 48 and the second end 50 may be brought closer together and an inner diameter of the resilient push tab 46 may decrease.

In some embodiments, the arced portion 52 may contact the catheter adapter 14 in response to the resilient push tab 46 being moved from the raised position to the depressed position. In some embodiments, the resilient push tab 46 may be coupled to the catheter adapter 14 proximal to the side port 40. In some embodiments, an outer surface of the catheter adapter 14 may include indents that may hold the first end 48 and the second end 50 in the raised position, but the first end 48 and the second end 50 may be configured to be removed from the indents in response to depression and pressing of the resilient push tab 46 by the clinician. In some embodiments, the catheter adapter 14 may not include the indents. In some embodiments, a septum 54 within the catheter adapter 14 may seal the proximal end 18 of the catheter adapter 14.

In some embodiments, a method may include inserting the catheter assembly 12 into vasculature of the patient. In some embodiments, the catheter assembly 12 may include the push tab 24 (discussed with respect to FIGS. 1A-3B) coupled to the catheter adapter 14. In some embodiments, the catheter assembly 12 may include the resilient push tab 46 coupled to the catheter adapter 14. In some embodiments, the push tab 24 or the resilient push tab 46 may be moveable between a raised position and a second position. In some embodiments, the push tab 24 or the resilient push tab 46 may be disposed in the raised position during insertion of the catheter assembly 12 into the vasculature of the patient. In some embodiments, insertion of the catheter assembly 12 into the vasculature of the patient may include initial insertion through the skin and a wall of a blood vessel and/or include advancement of the catheter 22 distally with respect to the introducer needle 41 in response to the introducer needle 41 being positioned within the vasculature.

In some embodiments, after the catheter assembly 12 is inserted into the vasculature of the patient, the method may include pushing down on the push tab 24 and/or the resilient push tab 46 to move the push tab 24 or the resilient push tab 46 from the raised position to the second position. In some embodiments, after pushing down on the push tab 24 or the resilient push tab 46 to move the push tab 24 or the resilient push tab 46 from the raised position to the second position, the method may include applying securement tape over the push tab 24 or the resilient push tab 46 with the push tab 24 or the resilient push tab 46 in the second position. In some embodiments, applying the securement tape over the push tab 24 or the resilient push tab 46 in the second position may prevent tenting of the securement tape and may increase stability of the catheter assembly 12 with respect to the patient.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter assembly, comprising:
a catheter adapter, comprising a distal end, a proximal end, and a lumen extending between the distal end and the proximal end; and
a push tab pivotally coupled to the catheter adapter, wherein the push tab is pivotally moveable between a raised position and a depressed position.

2. The catheter assembly of claim 1, wherein the push tab comprises a first end and a second end opposite the first end, wherein the first end is pivotally coupled to the catheter adapter, wherein the second end is further from the catheter adapter when the push tab is in the raised position than when the push tab is in the depressed position.

3. The catheter assembly of claim 2, wherein the second end is disposed in a first position in response to the push tab being in the raised position, wherein the second end is disposed in a second position in response to the push tab being in the depressed position, wherein the second position is proximal to the first position.

4. The catheter assembly of claim 1, wherein the push tab comprises two opposing protrusions, wherein the catheter adapter comprises two opposing indents, wherein the two opposing protrusions are disposed within the two opposing indents to pivotally couple the push tab to the catheter adapter.

5. The catheter assembly of claim 1, wherein in response to the push tab being in the raised position, the push tab is generally perpendicular to a longitudinal axis of the catheter adapter.

6. The catheter assembly of claim 1, wherein in response to the push tab being in the depressed position, the push tab is generally aligned with the longitudinal axis of the catheter adapter.

7. The catheter assembly of claim 1, wherein the push tab comprises an edge configured to contact the catheter adapter in response to the push tab pivotally moving from the depressed position to the raised position.

8. The catheter assembly of claim 1, wherein a proximal surface of the push tab is concave.

9. The catheter assembly of claim 1, wherein the catheter adapter comprises a side port disposed between the distal end and the proximal end, wherein the push tab is pivotally coupled to the catheter adapter proximal to the side port.

10. A catheter assembly, comprising:
a catheter adapter, comprising a distal end, a proximal end, and a lumen extending between the distal end and the proximal end; and
a resilient push tab coupled to the catheter adapter, wherein the resilient push tab comprises a first end, a second end, and an arced portion between the first end and the second end, wherein the resilient push tab is configured to move from a raised position to a depressed position, wherein the arced portion is further from the catheter adapter when the resilient push tab is in the raised position than when the resilient push tab is in the depressed position, wherein in response to the resilient push tab being in the raised position, the first end and the second end are biased outwardly by the catheter adapter, wherein in response to the resilient push tab moving from the raised position to the depressed position, the first end and the second end are brought closer together.

11. The catheter assembly of claim 10, wherein the arced portion contacts the catheter adapter in response to the resilient push tab being moved from the raised position to the depressed position.

12. The catheter assembly of claim 10, wherein the catheter adapter comprises a side port disposed between the distal end and the proximal end, wherein the resilient push tab is coupled to the catheter adapter proximal to the side port.

13. A method, comprising:
inserting a catheter assembly into vasculature of a patient, wherein the catheter assembly comprises:
a catheter adapter, comprising a distal end, a proximal end, and a lumen extending between the distal end and the proximal end; and
a push tab coupled to the catheter adapter, wherein the push tab is moveable between a raised position and a depressed position, wherein the push tab is disposed in the raised position;
after the catheter assembly is inserted into the vasculature of the patient, pushing down on the push tab to move the push tab from the raised position to the depressed position.

14. The method of claim 13, further comprising after pushing down on the push tab to move the push tab from the raised position to the depressed position, applying securement tape over the push tab with the push tab in the depressed position.

15. The method of claim 14, wherein the push tab is pivotally coupled to the catheter adapter, wherein the push tab is pivotally moveable between the raised position and the depressed position.

16. The catheter assembly of claim 15, wherein the push tab comprises a first end and a second end opposite the first end, wherein the first end is pivotally coupled to the catheter adapter, wherein the second end is further from the catheter adapter when the push tab is in the raised position than when the push tab is in the depressed position.

17. The catheter assembly of claim 16, wherein the push tab comprises two opposing protrusions, wherein the catheter adapter comprises two opposing indents, wherein the two opposing protrusions are disposed within the two opposing indents to pivotally couple the push tab to the catheter adapter.

18. The catheter assembly of claim 14, wherein the push tab comprises a resilient push tab, wherein the resilient push tab comprises a first end, a second end, and an arced portion between the first end and the second end, wherein the arced portion is further from the catheter adapter when the resilient push tab is in the raised position than when the resilient push tab is in the depressed position, wherein in response to the resilient push tab being in the raised position, the first end and the second end are biased outwardly by the catheter adapter, wherein in response to the resilient push tab moving from the raised position to the depressed position, the first end and the second end are brought closer together.

19. The catheter assembly of claim 18, wherein the arced portion contacts the catheter adapter in response to the resilient push tab being moved from the raised position to the depressed position.

20. The catheter assembly of claim 18, wherein the catheter adapter comprises a side port disposed between the distal end and the proximal end, wherein the resilient push tab is pivotally coupled to the catheter adapter proximal to the side port.
